(19) United States
(12) Patent Application Publication
(10) Pub. No.: US 2024/0325092 A1
(43) Pub. Date: Oct. 3, 2024

(54) SYSTEMS AND METHODS FOR POSE ESTIMATION OF IMAGING SYSTEM

(71) Applicant: Noah Medical Corporation, San Carlos, CA (US)

(72) Inventors: Tao Zhao, San Jose, CA (US); Changjiang Yang, Dayton, MD (US); Jason Joseph Hsu, Mountain View, CA (US); Zenan Zhang, San Mateo, CA (US); Frank Grillo, Los Altos, CA (US); Zhongming Shen, Sunnyvale, CA (US)

(21) Appl. No.: 18/738,282

(22) Filed: Jun. 10, 2024

Related U.S. Application Data
(63) Continuation of application No. PCT/US2022/054101, filed on Dec. 27, 2022.
(60) Provisional application No. 63/294,552, filed on Dec. 29, 2021.

(51) Int. Cl. A61B 34/20; A61B 1/00; A61B 6/02; A61B 6/12; A61B 34/10; A61B 34/30; A61B 90/00; G06T 7/73; G06T 15/00

(52) U.S. Cl. CPC A61B 34/20; A61B 1/00103; A61B 6/025; A61B 6/12; G06T 7/73; G06T 15/00; A61B 2034/107; A61B 2034/2065; A61B 2034/301; A61B 2090/3966; G06T 2207/10068; G06T 2207/10121; G06T 2207/30008; G06T 2207/30061; G06T 2207/30096; G06T 2207/30204; G06T 2210/41

(57) ABSTRACT

A method is provided for method for navigating a robotic endoscopic apparatus comprising: (a) providing a three-dimensional (3D) fiducial marker to a surgical field; (b) acquiring fluoroscopic image using a fluoroscopic imager wherein the fluoroscopic image contains the 3D fiducial marker, a body part and a portion of the robotic endoscopic apparatus placed inside of the body part; and (c) estimating a pose of the fluoroscopic imager based on the fluoroscopic image.

### REFERENCE

This application is a continuation of International Application No. PCT/US2022/054101, filed on Dec. 27, 2022, which claims priority to U.S. Provisional Patent Application No. 63/294,552, filed on Dec. 29, 2021, which is entirely incorporated herein by reference.

### BACKGROUND

Early diagnosis of lung cancer is critical. The five-year survival rate of lung cancer is around 18% which is significantly lower than next three most prevalent cancers: breast (90%), colorectal (65%), and prostate (99%). A total of 142,000 deaths were recorded in 2018 due to lung cancer.

In general, a typical lung cancer diagnosis and surgical treatment process can vary drastically, depending on the techniques used by healthcare providers, the clinical protocols, and the clinical sites. The inconsistent process can delay the diagnosis of the cancer as well as imposing a high cost on the patient and the health care system.

These medical procedures such as endoscopy (e.g., bronchoscopy) may involve accessing and visualizing the inside of a patient's lumen (e.g., airways) for diagnostic and/or therapeutic purposes. During a procedure, a flexible tubular tool such as, for example, an endoscope, may be inserted into the patient's body and an instrument can be passed through the endoscope to a tissue site identified for diagnosis and/or treatment.

### SUMMARY OF THE INVENTION

Endoscopes comprise vast applications in diagnosis and treatment of various conditions, such as medical conditions (e.g., early lung cancer diagnosis and treatment). An endoscopy navigation system may use different sensing modalities (e.g., camera imaging data, electromagnetic (EM) position data, robotic position data, etc. In some cases, the navigation approach may depend on an initial estimate of where the tip of the endoscope is with respect to the airway to begin tracking the tip of the endoscope. Some endoscopy techniques may involve a three-dimensional (3D) model of a patient's anatomy (e.g., CT image), and guide navigation using an EM field and position sensors.

In some cases, 3D image of a patient's anatomy may be taken one or more times for various purposes. For instance, prior to a medical procedure, 3D model of a patient anatomy may be created to identify the target location. In some cases, the precise alignment (e.g., registration) between the virtual space of the 3D model, the physical space of the patient's anatomy represented by the 3D model, and the EM field may be unknown. As such, prior to generating a registration, endoscope positions within the patient's anatomy cannot be mapped with precision to corresponding locations within the 3D model. In another instance, during surgical operation, 3D imaging may be performed to update/confirm the location of the target (e.g., lesion) in the case of movement of the target issue or lesion. In some cases, to assist with reaching the target tissue location, the location and movement of the medical instruments may be registered with intra-operative images of the patient anatomy. With the image-guided instruments registered to the images, the instruments may navigate natural or surgically created passageways in anatomical systems such as the lungs, the colon, the intestines, the kidneys, the heart, the circulatory system, or the like. In some instances, after the medical instrument (e.g., needle, endoscope) reaches the target location or after a surgical operation is completed. 3D imaging may be performed to confirm the instrument or operation is at the target location.

In some cases, fluoroscopic imaging systems may be used to determine the location and orientation of medical instruments and patient anatomy within the coordinate system of the surgical environment. In order for the imaging data to assist in correctly localizing the medical instrument, the coordinate system of the imaging system may be needed for reconstructing the 3D model. Systems and methods herein may provide pose estimation of the imaging system to more reliably localize and navigate medical instruments within the surgical environment.

In an aspect, a method for navigating a robotic endoscopic apparatus is provided. The method comprises: (a) acquiring one or more fluoroscopic images using a fluoroscopic imager, wherein the one or more fluoroscopic images contain an image of a fiducial marker, a body part and a portion of the robotic endoscopic apparatus placed inside of the body part, wherein the fiducial marker is a three-dimensional (3D) marker: (b) estimating a pose of the fluoroscopic imager based at least in part on image of the fiducial marker in the one or more fluoroscopic images; and (c) reconstruction a 3D fluoroscopic image based at least in part on the pose estimated in (b).

In a related yet separate aspect, the present disclosure provides a non-transitory computer-readable storage medium including instructions that, when executed by one or more processors, cause the one or more processors to perform operations. The operations comprise: (a) acquiring one or more fluoroscopic images using a fluoroscopic imager, wherein the one or more fluoroscopic images contain an image of a fiducial marker, a body part and a portion of the robotic endoscopic apparatus placed inside of the body part, wherein the fiducial marker is a three-dimensional (3D) marker: (b) estimating a pose of the fluoroscopic imager based at least in part on image of the fiducial marker in the one or more fluoroscopic images; and (c) reconstruction a 3D fluoroscopic image based at least in part on the pose estimated in (b).

In some embodiments, the fiducial marker is deployed to a surgical field and wherein at least one of the one or more fluoroscopic images is acquired at an angle that is substantially aligned to a patient bed in the surgical field. In some embodiments, the fiducial marker is an anatomical structure in the body part. In some cases, the fiducial marker is a bone structure.

In some embodiments, the robotic endoscopic apparatus is disposable. In some embodiments, the method further comprises confirming the portion of the robotic endoscopic apparatus is inside a target tissue in the body part based on the 3D fluoroscopic image or updating a location of a target tissue based on the 3D fluoroscopic image.

In some embodiments, the fiducial marker comprises a first 3D marker and a second 3D marker. In some cases, the pose of the fluoroscopic imager is estimated based on a first pose estimated using the first 3D marker and a second pose estimated using the second 3D marker. In some instances, the pose of the fluoroscopic imager is a weighted average of the first pose and the second pose. In some instances, the pose of the fluoroscopic imager is estimated using an optimization algorithm and wherein the first pose or the second pose is used as an initial solution of the optimization algorithm. In some cases, the first fiducial marker is an anatomical structure and the second fiducial marker is an artificial 3D marker. In some cases, the fiducial marker includes a first component located at a patient bed and a second component located on a patient.

In some embodiments, the method further comprises receiving a measured pose of the fluoroscopic imager based on location sensor data and fusing the measured pose with the pose estimated in (b).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example workflow of lung cancer diagnosis enabled by the robotic bronchoscopy system described herein.

FIG. 2A shows examples of robotic bronchoscopy systems, in accordance with some embodiments of the invention.

FIG. 2B shows different views of an example robotic bronchoscopy system, in accordance with some embodiments of the invention.

FIG. 3 shows an example of a fluoroscopy (tomosynthesis) imaging system.

FIG. 4 shows a C-arm fluoroscopy (tomosynthesis) imaging system in different (rotation) poses while taking images of a subject.

FIG. 5 shows an example image of X-ray visible fiducial markers.

FIG. 6 shows an example of a fluoroscopic imaging system arranged near a patient to obtain fluoroscopic images of the patient.

FIG. 7 shows an example of a tomosynthesis image with the bronchoscope is reaching a target lesion site.

FIG. 8 shows an example of using natural high contrast structures (e.g., bones) as fiducial landmarks.

FIG. 9 schematically illustrates an intelligent fusion framework for dynamically fusing and processing real-time sensory data to generate a pose estimation for the imaging device.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

While exemplary embodiments will be primarily directed at a bronchoscope, one of skill in the art will appreciate that this is not intended to be limiting, and the devices described herein may be used for other therapeutic or diagnostic procedures and in other anatomical regions of a patient's body such as a digestive system, including but not limited to the esophagus, liver, stomach, colon, urinary tract, or a respiratory system, including but not limited to the bronchus, the lung, and various others.

The embodiments disclosed herein can be combined in one or more of many ways to provide improved diagnosis and therapy to a patient. The disclosed embodiments can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

Although the treatment planning and definition of diagnosis or surgical procedures as described herein are presented in the context of pulmonary diagnosis or surgery, the methods and apparatus as described herein can be used to treat any tissue of the body and any organ and vessel of the body such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat.

Whenever the term “at least.” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than.” “less than,” or “less than or equal to” precedes the first numerical value in a series of two or more numerical values, the term “no more than.” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU) or a microcontroller), digital signal processors (DSPs), a field programmable gate array (FPGA) and/or one or more Advanced RISC Machine (ARM) processors. In some cases, the one or more processors may be operatively coupled to a non-transitory computer readable medium. The non-transitory computer readable medium can store logic, code, and/or program instructions executable by the one or more processors unit for performing one or more steps. The non-transitory computer readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

As used herein, the terms distal and proximal may generally refer to locations referenced from the apparatus, and can be opposite of anatomical references. For example, a distal location of a bronchoscope or catheter may correspond to a proximal location of an elongate member of the patient, and a proximal location of the bronchoscope or catheter may correspond to a distal location of the elongate member of the patient.

A system as described herein, includes an elongate portion or elongate member such as a catheter. The terms “elongate member”, “catheter”, “bronchoscope” are used interchangeably throughout the specification unless contexts suggest otherwise. The elongate member can be placed directly into the body lumen or a body cavity. In some embodiments, the system may further include a support apparatus such as a robotic manipulator (e.g., robotic arm) to drive, support, position or control the movements and/or operation of the elongate member. Alternatively or in addition to, the support apparatus may be a hand-held device or other control devices that may or may not include a robotic system. In some embodiments, the system may further include peripheral devices and subsystems such as imaging systems that would assist and/or facilitate the navigation of the elongate member to the target site in the body of a subject. Such navigation may require a registration process which will be described later herein.

In some embodiments of the present disclosure, a robotic bronchoscopy system is provided for performing surgical operations or diagnosis with improved performance at low cost. For example, the robotic bronchoscopy system may comprise a steerable catheter that can be entirely disposable. This may beneficially reduce the requirement of sterilization which can be high in cost or difficult to operate, yet the sterilization or sanitization may not be effective. Moreover, one challenge in bronchoscopy is reaching the upper lobe of the lung while navigating through the airways. In some cases, the provided robotic bronchoscopy system may be designed with capability to navigate through the airway having a small bending curvature in an autonomous or semi-autonomous manner. The autonomous or semi-autonomous navigation may require a registration process as described later herein. Alternatively, the robotic bronchoscopy system may be navigated by an operator through a control system with vision guidance.

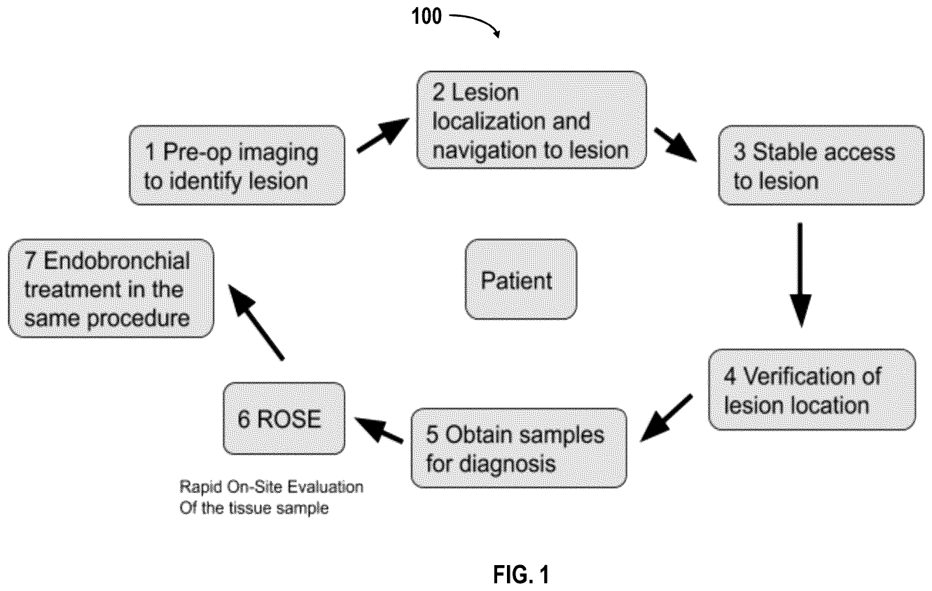

A typical lung cancer diagnosis and surgical treatment process can vary drastically, depending on the techniques used by healthcare providers, the clinical protocols, and the clinical sites. The inconsistent processes may cause delay to diagnose lung cancers in early stage, high cost of healthcare system and the patients to diagnose and treat lung cancers, and high risk of clinical and procedural complications. The provided robotic bronchoscopy system may allow for standardized early lung cancer diagnosis and treatment. FIG. 1 shows an example workflow 100 of standardized lung cancer diagnosis enabled by the robotic bronchoscopy system described herein.

As illustrated in FIG. 1, in some cases, a pre-operative imaging may be performed to identify lesions, and/or to identify the airways which will be used for registration and navigation during the procedure. Any suitable imaging modalities such as magnetic resonance (MR), positron emission tomography (PET), X-ray, computed tomography (CT) and ultrasound may be used to identify lesions or regions of interest. For instance, a patient with suspect lung cancer may be administered a pre-operative CT scan and suspect lung nodules may be identified in the CT images. The pre-operative imaging process can be performed prior to the bronchoscopy. The CT images may be analyzed to generate a map to guide the navigation of the robotic bronchoscope at the time of bronchoscopy. For example, the lesion or the region of interest (ROI) may be segmented on the images. When the lung is under imaging, the passage or pathway to the lesion may be highlighted on the reconstructed images for planning a navigation path. The reconstructed images may guide the navigation of the robotic bronchoscope to the target tissue or target site. In some cases, the navigation path may be pre-planned using 3D image data. For instance, the catheter may be advanced toward the target site under a robotic control of the robotic bronchoscope system. The catheter may be steered or advanced towards the target site in a manual manner, an autonomous manner, or a semi-autonomous manner. In an example, the movement of the catheter may be image guided such that the insertion and/or steering direction may be controlled automatically.

In some cases, the lesion location in the pre-operative imaging may not be accurate due to various reasons, such as CT to body divergence (CT2BD). CT2BD is the discrepancy of the electronic virtual target and the actual anatomic location of the peripheral lung lesion. CT2BD can occur for a variety of reasons including atelectasis, neuromuscular weakness due to anesthesia, tissue distortion from the catheter system, bleeding, ferromagnetic interference, and perturbations in anatomy such as pleural effusions. It is desirable to provide intra-operative real time correction for CT2BD. For instance, the lesion location may be verified prior to a surgical procedure (e.g., biopsy or treatment) during the operation or when the tip of the endoscope is within proximity of the target. The accurate location of the lesion may be verified or updated with aid of the robotic bronchoscopy system. For instance, the bronchoscopy system may provide interface to imaging modalities such as fluoroscopy to provide in vivo real-time imaging of the target site and the surrounding areas to locate the lesion. In an example, a C-arm or O-arm fluoroscopic imaging system may be used to generate a tomosynthesis or Cone Beam CT image for verifying or updating the location of the lesion. Upon confirmation or updating the lesion location, the process may proceed to the surgical procedures such as biopsy, and various surgical tools such as biopsy tools, brushes or forceps may be inserted into the working channel of the catheter to perform biopsy or other surgical procedures manually or automatically. In some cases, another fluoroscopy may be performed to confirm the tools have reached the target site.

In some cases, samples of the lesion or any other target tissue may be obtained by the tools inserted through the working channel of the catheter. The system allows for camera visualization to be maintained throughout the procedure, including during the insertion of tools through the working channel. In some cases, the tissue sample may be rapidly evaluated on-site by a rapid on-site evaluation process to determine whether repetition of the tissue sampling is needed, or to decide further action. In some cases, the rapid on-site evaluation process may also provide a quick analysis on the tissue sample to determine the following surgical treatment. For instance, if the tissue sample is determined to be malignant as a result of the rapid on-site evaluation process, a manual or robotic treatment instrument may be inserted through the working channel of the robotic bronchoscope and perform endobronchial treatment of the lung cancer. This beneficially allows for diagnosis and treatment being performed in one session thereby providing targeted, painless, and fast treatment of early-stage lung cancer.

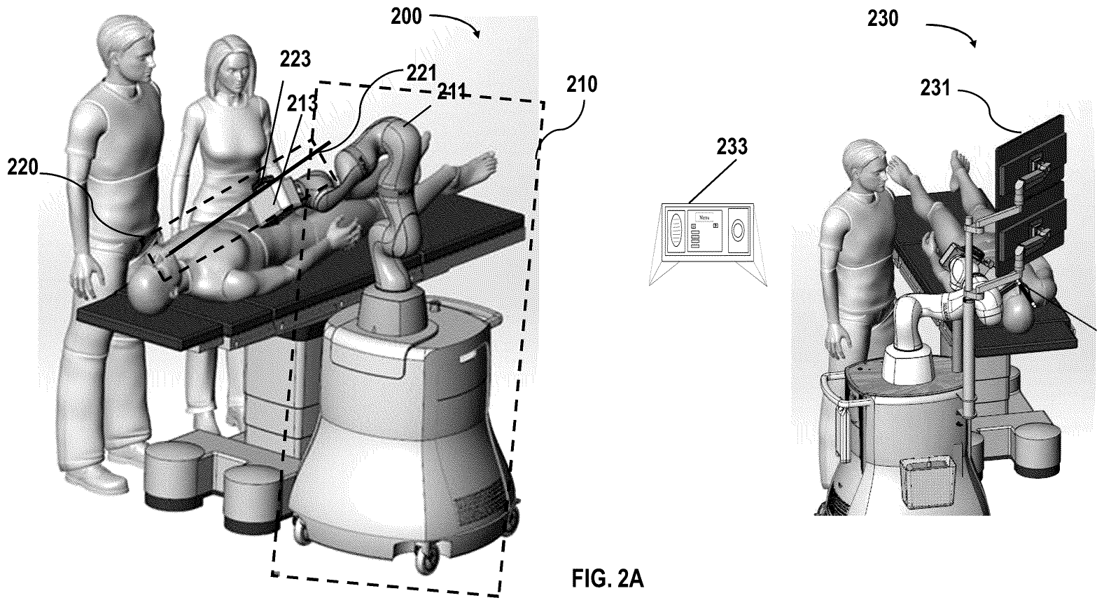

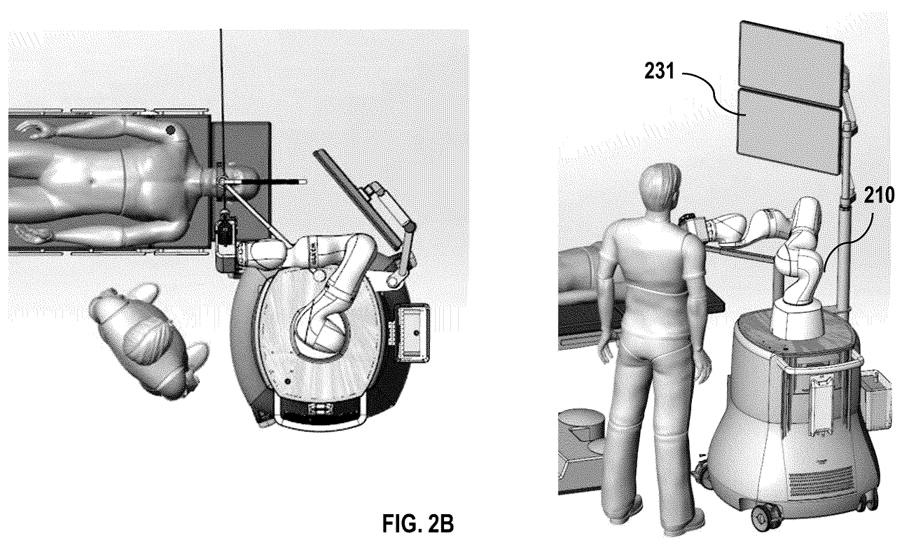

FIGS. 2A and 2B show examples of robotic bronchoscopy system 200, 230, in accordance with some embodiments of the invention. As shown in FIG. 2A, the robotic bronchoscopy system 200 may comprise a steerable catheter assembly 220 and a robotic support system 210, for supporting or carrying the steerable catheter assembly. The steerable catheter assembly can be a bronchoscope. In some embodiments, the steerable catheter assembly may be a single-use robotic bronchoscope. In some embodiments, the robotic bronchoscopy system 200 may comprise an instrument driving mechanism 213 that is attached to the arm of the robotic support system. The instrument driving mechanism may be provided by any suitable controller device (e.g., hand-held controller) that may or may not include a robotic system. The instrument driving mechanism may provide mechanical and electrical interface to the steerable catheter assembly 220. The mechanical interface may allow the steerable catheter assembly 220 to be releasably coupled to the instrument driving mechanism. For instance, a handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool.

The steerable catheter assembly 220 may comprise a handle portion 223 that may include components configured to processing image data, provide power, or establish communication with other external devices. For instance, the handle portion 223 may include a circuitry and communication elements that enables electrical communication between the steerable catheter assembly 220 and the instrument driving mechanism 213, and any other external system or devices. In another example, the handle portion 223 may comprise circuitry elements such as power sources for powering the electronics (e.g., camera and LED lights) of the endoscope. In some cases, the handle portion may be in electrical communication with the instrument driving mechanism 213 via an electrical interface (e.g., printed circuit board) so that image/video data and/or sensor data can be received by the communication module of the instrument driving mechanism and may be transmitted to other external devices/systems. Alternatively or in addition to, the instrument driving mechanism 213 may provide a mechanical interface only. The handle portion may be in electrical communication with a modular wireless communication device or any other user device (e.g., portable/hand-held device or controller) for transmitting sensor data and/or receiving control signals. Details about the handle portion are described later herein.

The steerable catheter assembly 220 may comprise a flexible elongate member 211 that is coupled to the handle portion. In some embodiments, the flexible elongate member may comprise a shaft, steerable tip and a steerable section. The steerable catheter assembly may be a single use robotic bronchoscope. In some cases, only the elongate member may be disposable. In some cases, at least a portion of the elongate member (e.g., shaft, steerable tip, etc.) may be disposable. In some cases, the entire steerable catheter assembly 220 including the handle portion and the elongate member can be disposable. The flexible elongate member and the handle portion are designed such that the entire steerable catheter assembly can be disposed of at low cost. Details about the flexible elongate member and the steerable catheter assembly are described later herein.

In some embodiments, the provided bronchoscope system may also comprise a user interface. As illustrated in the example system 230, the bronchoscope system may include a treatment interface module 231 (user console side) and/or a treatment control module 233 (patient and robot side). The treatment interface module may allow an operator or user to interact with the bronchoscope during surgical procedures. In some embodiments, the treatment control module 233 may be a hand-held controller. The treatment control module may, in some cases, comprise a proprietary user input device and one or more add-on elements removably coupled to an existing user device to improve user input experience. For instance, physical trackball or roller can replace or supplement the function of at least one of the virtual graphical element (e.g., navigational arrow displayed on touchpad) displayed on a graphical user interface (GUI) by giving it similar functionality to the graphical element which it replaces. Examples of user devices may include, but are not limited to, mobile devices, smartphones/cellphones, tablets, personal digital assistants (PDAs), laptop or notebook computers, desktop computers, media content players, and the like. Details about the user interface device and user console are described later herein.

FIG. 2B shows different views of a bronchoscope system. The user console 231 may be mounted to the robotic support system 210. Alternatively or in addition to, the user console or a portion of the user console (e.g., treatment interface module) may be mounted to a separate mobile cart.

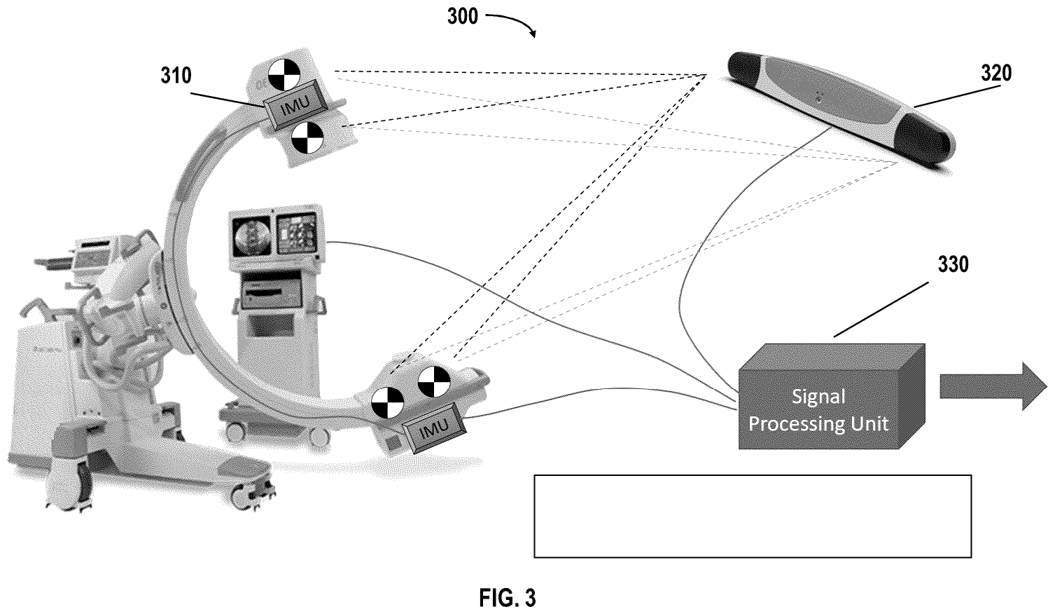

In one aspect, a robotic endoluminal platform is provided. In some cases, the robotic endoluminal platform may be a bronchoscopy platform. The platform may be configured to perform one or more operations consistent with the method described in FIG. 1. FIG. 3 shows an example of a robotic endoluminal platform and its components or subsystems, in accordance with some embodiments of the invention. In some embodiments, the platform may comprise a robotic bronchoscopy system and one or more subsystems that can be used in combination with the robotic bronchoscopy system of the present disclosure.

In some embodiments, the one or more subsystems may include imaging systems such as a fluoroscopy imaging system for providing real-time imaging of a target site (e.g., comprising lesion). Multiple 2D fluoroscopy images may be used to create tomosythesis or Cone Beam CT (CBCT) reconstruction to better visualize and provide 3D coordinates of the anatomical structures. Tomosynthesis may refer to limited angle tomography in contrast to full-angle (e.g., 180-degree tomography). In some cases, the tomosynthesis image reconstruction may comprise generating a 3D volume with a combination of X-ray projections images acquired at different angles (acquired by any type of C-arm systems). FIG. 3 shows an example of a fluoroscopy (tomosynthesis) imaging system 300. For example, the fluoroscopy (tomosynthesis) imaging system may perform accurate lesion location tracking or verification before or during surgical procedure as described in FIG. 1. In some cases, lesion location may be tracked based on location data about the fluoroscopy (tomosynthesis) imaging system/station (e.g., C arm) and image data captured by the fluoroscopy (tomosynthesis) imaging system. The lesion location may be registered with the coordinate frame of the robotic bronchoscopy system.

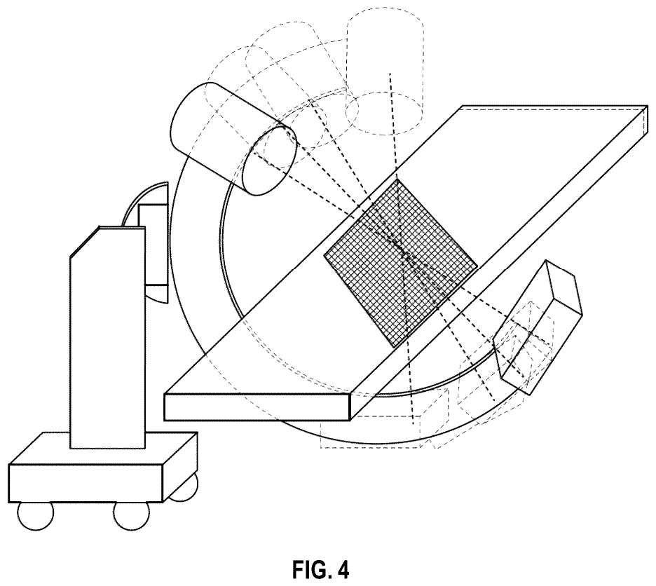

In some cases, a location, pose or motion of the fluoroscopy imaging system may be measured/estimated to register the coordinate frame of the image to the robotic bronchoscopy system, or for constructing the 3D model/image. The pose or motion of the fluoroscopy (tomosynthesis) imaging system may be measured using any suitable motion/location sensors 310 disposed on the fluoroscopy (tomosynthesis) imaging system. The motion/location sensors may include, for example, inertial measurement units (IMUs)), one or more gyroscopes, velocity sensors, accelerometers, magnetometers, location sensors (e.g., global positioning system (GPS) sensors), vision sensors (e.g., imaging devices capable of detecting visible, infrared, or ultraviolet light, such as cameras), proximity or range sensors (e.g., ultrasonic sensors, lidar, time-of-flight or depth cameras), altitude sensors, attitude sensors (e.g., compasses) and/or field sensors (e.g., magnetometers, electromagnetic sensors, radio sensors). In some cases, the one or more sensors for tracking the motion and location of the fluoroscopy (tomosynthesis) imaging station may be disposed on the imaging station or be located remotely from the imaging station, such as a wall-mounted camera 320. FIG. 4 shows a C-arm fluoroscopy (tomosynthesis) imaging system in different (rotation) poses while taking images of a subject. The various poses may be captured by the one or more sensors as described above.

In some embodiments, a location of lesion may be segmented in the image data captured by the fluoroscopy (tomosynthesis) imaging system with aid of a signal processing unit 330. One or more processors of the signal processing unit may be configured to further overlay treatment locations (e.g., lesion) on the real-time fluoroscopic image/video. For example, the processing unit may be configured to generate an augmented layer comprising augmented information such as the location of the treatment location or target site. In some cases, the augmented layer may also comprise graphical marker indicating a path to this target site. The augmented layer may be a substantially transparent image layer comprising one or more graphical elements (e.g., box, arrow, etc.). The augmented layer may be superposed onto the optical view of the optical images or video stream captured by the fluoroscopy (tomosynthesis) imaging system, and/or displayed on the display device. The transparency of the augmented layer allows the optical image to be viewed by a user with graphical elements overlay on top of. In some cases, both the segmented lesion images and an optimum path for navigation of the elongate member to reach the lesion may be overlaid onto the real time tomosynthesis images. This may allow operators or users to visualize the accurate location of the lesion as well as a planned path of the bronchoscope movement. In some cases, the segmented and reconstructed images (e.g., CT images as described elsewhere) provided prior to the operation of the systems described herein may be overlaid on the real time images.

In some embodiments, the one or more subsystems of the platform may comprise one or more treatment subsystems such as manual or robotic instruments (e.g., biopsy needles, biopsy forceps, biopsy brushes) and/or manual or robotic therapeutical instruments (e.g., RF ablation instrument, Cryo instrument, Microwave instrument, and the like).

In some embodiments, the one or more subsystems of the platform may comprise a navigation and localization subsystem. The navigation and localization subsystem may be configured to construct a virtual airway model based on the pre-operative image (e.g., pre-op CT image or tomosynthesis). The navigation and localization subsystem may be configured to identify the segmented lesion location in the 3D rendered airway model and based on the location of the lesion, the navigation and localization subsystem may generate an optimal path from the main bronchi to the lesions with a recommended approaching angle towards the lesion for performing surgical procedures (e.g., biopsy).

At a registration step before driving the bronchoscope to the target site, the system may align the rendered virtual view of the airways to the patient airways. Image registration may consist of a single registration step or a combination of a single registration step and real-time sensory updates to registration information. The registration process may include finding a transformation that aligns an object (e.g., airway model, anatomical site) between different coordinate systems (e.g., EM sensor coordinates and patient 3D model coordinates based on pre-operative CT imaging). For example, in real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter) may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively registered to the patient anatomy (e.g., 3D model) to determine the registration transformation that aligns a single location in the coordinate system with a position in the pre-operative model of the patient's anatomy.

Once registered, all airways may be aligned to the pre-operative rendered airways. During robotic bronchoscope driving towards the target site, the location of the bronchoscope inside the airways may be tracked and displayed. In some cases, location of the bronchoscope with respect to the airways may be tracked using positioning sensors. Other types of sensors (e.g., camera) can also be used instead of or in conjunction with the positioning sensors using sensor fusion techniques. Positioning sensors such as electromagnetic (EM) sensors may be embedded at the distal tip of the catheter and an EM field generator may be positioned next to the patient torso during procedure. The EM field generator may locate the EM sensor position in 3D space or may locate the EM sensor position and orientation in 5D or 6D space. This may provide a visual guide to an operator when driving the bronchoscope towards the target site.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively registered to the patient anatomy (e.g., 3D model) in order to determine the registration transformation that aligns a single location in the coordinate system with a position in the pre-operative model of the patient's anatomy.

### Pose Estimation of the Imaging System

In some embodiments, the platform herein may utilize fluoroscopic imaging systems to determine the location and orientation of medical instruments and patient anatomy within the coordinate system of the surgical environment. In particular, the systems and methods herein may employ a mobile C-arm fluoroscopy as a low-cost and mobile real-time qualitative assessment tool. C-arms, however, may not be widely accepted for applications involving quantitative assessments, mainly due to the lack of reliable and low-cost position/pose tracking methods, as well as adequate calibration and registration techniques. The present disclosure provides various pose estimation and/or tracking methods that can be retrofitted to any conventional C-arm imaging system.

Fluoroscopy is an imaging modality that obtains real-time moving images of patient anatomy, and medical instruments. Fluoroscopic systems may include C-arm systems which provide positional flexibility and are capable of orbital, horizontal, and/or vertical movement via manual or automated control. Fluoroscopic image data from multiple viewpoints (i.e., with the fluoroscopic imager moved among multiple locations) in the surgical environment may be compiled to generate two-dimensional or three-dimensional tomographic images. When using a fluoroscopic imager system that include a digital detector (e.g., a flat panel detector), the generated and compiled fluoroscopic image data may be permit the sectioning of planar images in parallel planes according to tomosynthesis imaging techniques.

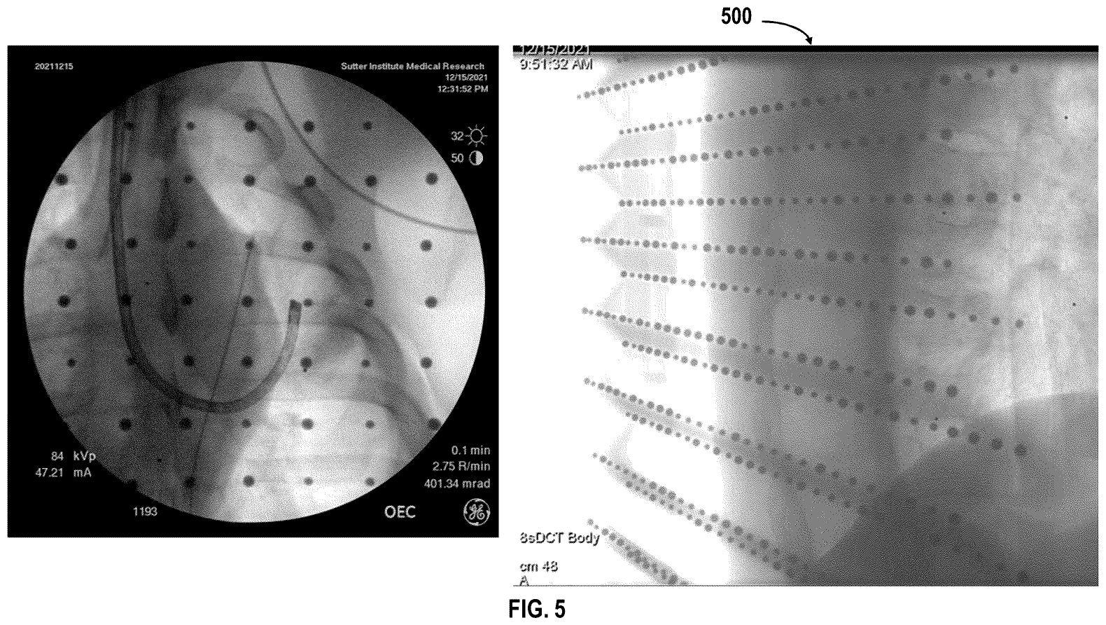

Traditional fluoroscopic imager movement may be constrained to a limited angular range. As shown in FIG. 4, current tomosynthesis imaging quality may be constrained to the imitated rotation range (e.g., 60 degree) beyond which the quality of the 3D reconstructed image (i.e., tomosynthesis) may be degraded due to the invisibility of the planar or quasi-planar fiducial markers. As shown in FIG. 5, conventional fiducial markers may include a matrix of ball bearings that can be segmented easily and analyzed to register the unique location of each ball bearing on any radiographic images within a limited angle range (e.g., 60 degree). For instance, pairs of radiographs acquired at different angles may be analyzed to reconstruct the three-dimensional locations of ball bearings based on the segmented locations of the ball bearings and the corresponding intrinsic and extrinsic parameters found from gantry calibration. The results are then used to determine the corresponding pose of the imaging system which is further used to reconstruct 3D image. However, as shown in the example 500, as the matrix of ball bearings are substantially disposed on the same plane and when the imager is rotated to a large angle that is substantially aligned to the markers' plane, the markers may not be visible/discernable in the 2D image resulting in degraded quality of the 3D image.

The present disclosure provides various methods to track pose of the fluoroscopic imager and allows for an enlarged rotation angle (e.g., at least 120 degree, 130 degree, 140 degree, 150 degree, 160 degree, 170 degree, 180 degree or greater) of the C-arm fluoroscopy system. The fluoroscopic imager may be moved around in the three dimensional surgical space to capture image data that is tomographically combined to generate optimal images of a region of interest in the patient anatomy.

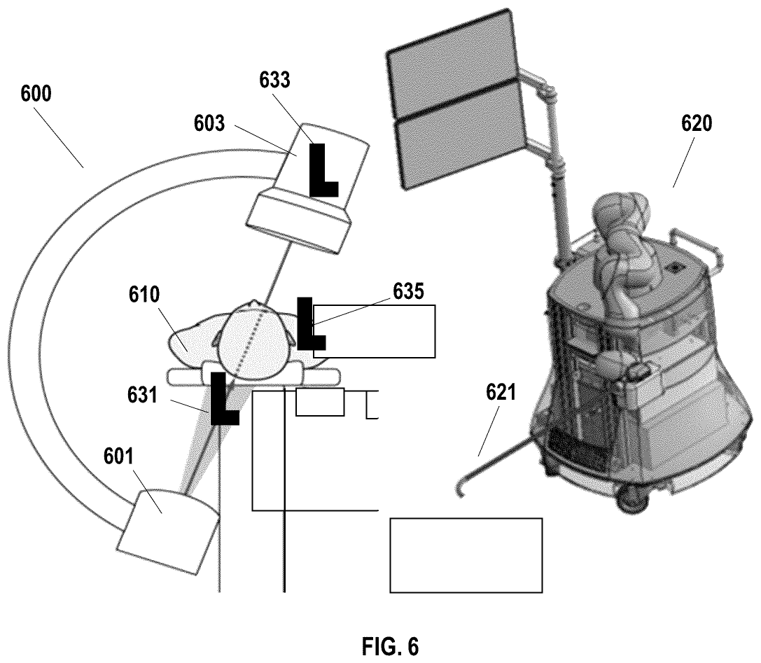

In particular, the pose estimation or tracking methods for the C-arm fluoroscopy system may beneficially allow for three-dimensional localization of the structural landmarks of the patient's anatomy based on multiple radiographic views. FIG. 6 shows an example of a fluoroscopic imaging system 600 arranged near a patient 610 to obtain fluoroscopic images of the patient. The system may be, for example, a mobile C-arm fluoroscopic imaging system. In some embodiments, the system may be a multi-axis fluoroscopic imaging system.

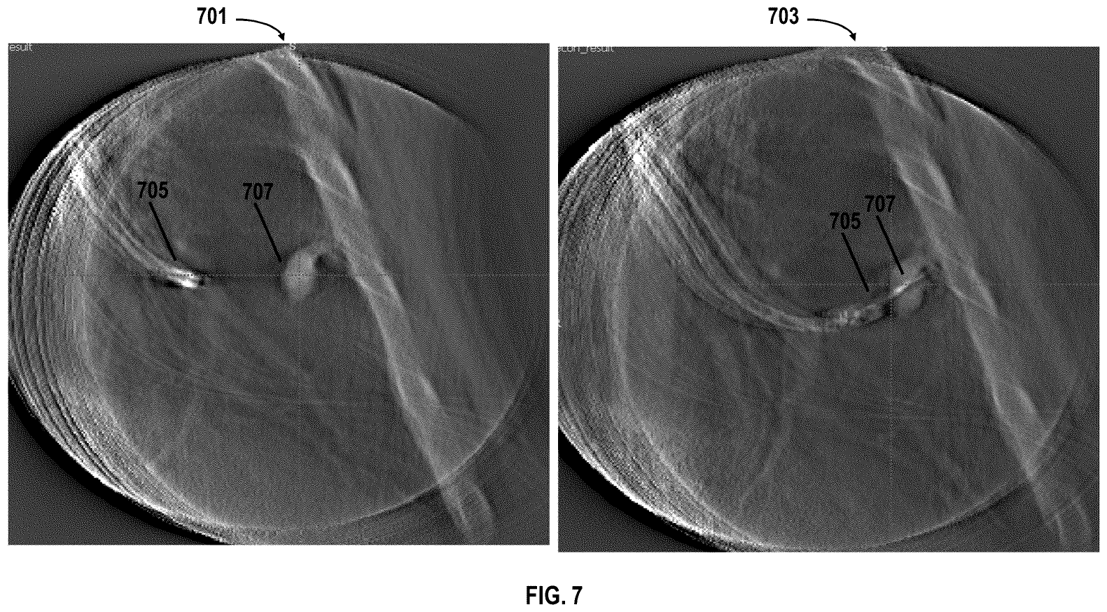

The fluoroscopic image may be acquired prior to inserting a catheter 621 into the patient body. In some cases, a physician may want to verify a lesion location right before navigating the catheter to the lesion site. In some cases, the fluoroscopic image may be acquired while the catheter 621 is extended within the patient 610. FIG. 7 shows an example of a fluoroscopic image 701 with the catheter 705 is reaching a target lesion site 707. The fluoroscopic image 701 may be utilized to update a precise location of the target tissue (e.g., target lesion) with respect to the catheter. In some cases, the fluoroscopic image 703 may be acquired after the catheter 705 reached the target site 707 to confirm the location of the catheter with respect to the target location in real-time. In some cases, the fluoroscopic image 703 may be used to confirm whether a tool (e.g., needle extended out of the endoscope tip) is inside of a target tissue (e.g., lesion).

Referring back to FIG. 6, the C-arm imaging system 600 may comprise a source 601 (e.g., an X-ray source) and a detector 603 (e.g., an X-ray detector or X-ray imager). The X-ray detector may generate an image representing the intensities of received x-rays. The imaging system 600 may reconstruct 3D image based on multiple 2D image acquired from a wide range of angels. In some cases, the rotation angle range may be at least 120-degree, 130-degree, 140-degree, 150-degree, 160-degree. 170-degree, 180-degree or greater. In some cases, the 3D image may be generated based on a pose of the X-ray imager. The pose may be reconstructed or measured by a sensing device. The present disclosure provides various methods to augment the pose estimation capability of a conventional C-arm system that may not have the pose measurement available.

In some embodiments, a pose of the X-ray imager may be estimated using improved fiducial markers. Instead of using 2D pattern as fiducial marker, the present disclosure may provide 3D fiducial marker such that the marker is visible and discernable in a wide range of angles. For example, a 3D fiducial marker 631 may be located within the view of the imaging system such that the fiducial marker is always discernable when the imager rotates in the wide angle range. In particular, the 3D fiducial marker can be clearly visualized even when the imager rotates to an angle substantially aligned with the patient bed plane. This beneficially allows for reconstruction of 3D image using 2D images acquired from a wide angle range that would have not been available utilizing 2D fiducial markers.

The 3D marker herein may be radiopaque. For instance, the 3D marker may appear white and visible in the radiographic image. The fiducial marker(s) may have any suitable 3D shape (non-isotropic) or pattern such that a projection of the fiducial mark(s) corresponding to a view/angle is discernable from that of another view/angle. As shown in the example, the 3D marker is L shaped. However, the 3D marker can be in any other non-isotropic shape or pattern.

The fiducial marker(s) may be located above, aside, or under the patient 610 (e.g., on the patient bed). In some cases, the fiducial marker(s) may be placed on a garment worn by the patient (e.g., ball bearings in/on cloth worn by patient). In some cases, the fiducial marker(s) may be sticked to the patient body. For instance, a patch, band with ball bearings may be applied to the patient. In some cases, a 3D fiducial marker may comprise multiple parts located not on the same planar plane. For instance, the 3D marker may comprise a first part 635 placed on a body part of the patient (e.g., on cloth worn by the patient, located on the chest, etc.) and a second part 631 (e.g., pattern array) located at the patient bed. The first part and the second part may or may not be a 3D marker (e.g., array of 2D pattern or 2D shape) but they may collectively form a 3D marker when they are placed not on the same plane.

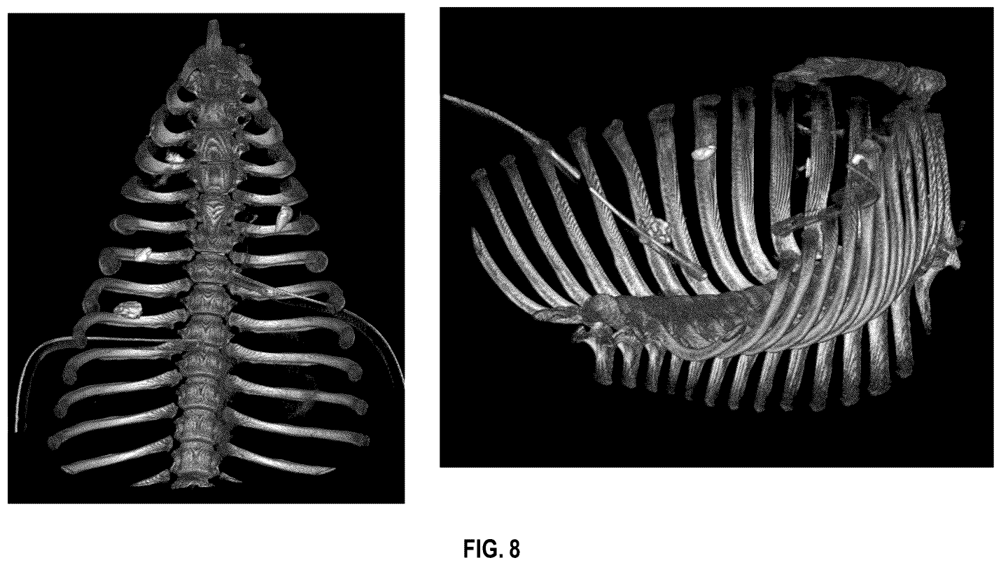

The fiducial markers may be artificial markers created and/or added to the platform. Alternatively, an existing features may be utilized as 3D fiducial markers. In some cases, the fiducial marker may comprise an anatomical landmark of the patient 610. For example, as shown in FIG. 8, one or more bone structures (e.g., ribs) of the patient may be used as marker to recover the pose of the imager when taken from multiple viewpoints. For instance, the one or more bone structures in the 2D fluoroscopic images acquired from multiple angles may be segmented and matched (e.g., blob detection or other computer vision techniques) to identify the same bone structure across multiple 2D fluoroscopic images. Next, an accurate camera pose and camera parameters may be recovered or estimated for the tomosynthesis image reconstruction. For instance, pose estimation may include recovering rotation and translation of the camera (camera poses) by minimizing reprojection error from 3D-2D point correspondences. In some cases, an optimization algorithm may be used to refine camera calibration parameters by minimizing the reprojection error. The optimization algorithm may be a least squares algorithm, such as the global Levenberg-Marquardt optimization. Recovering the camera pose may further include estimating the pose of a calibrated camera given a set of n 3D points in the world and their corresponding 2D projections in the images. In this example, the points and 2D projections are based on points of the anatomical/bone structures. The camera pose may include 6 degrees-of-freedom with rotation (e.g., roll, pitch, yaw) and 3D translation of the camera with respect to the world. Perspective-n-Point (PnP) pose computation may be used to recover the camera poses from n pairs of point correspondences. In some cases, n=3, therefore, the minimal form of the PnP problem is P3P, which may be solved with three point correspondences. For each tomosynthesis frame, there may be a plurality of marker matches (e.g., a plurality of bone structures) and a RANSAC variant of the PnP solver may be used for the camera pose estimation. Once estimated, the pose may be further refined by minimizing the reprojection error using a non-linear minimization method and starting from the initial pose estimate with the PnP solver.

In some cases, performing the camera pose estimation for tomosynthesis reconstruction may include generating undistorted images (e.g., from the robotic bronchoscopy system). The undistorted images may be generated by image pre-processing such as image inpainting, etc. In some cases, the undistorted image may be normalized using a normalization algorithm. For example, the undistorted image may be normalized using a logarithmic normalization algorithm, such as Beer's Law:

$$- log \frac{b + δ}{max ( b ) + δ} ,$$

where b is the input image and δ is an offset to avoid a zero logarithm. In some cases, projection matrices (e.g., estimated camera pose matrices) and/or physical parameters (e.g., size, resolution, position, volume, geometry, etc.) of the tomosynthesis reconstruction may be obtained. Inputs of one or more of the normalized images, the projection matrices, or the physical parameters, may enable generation of a reconstructed volume for the tomosynthesis reconstruction.

In exemplary implementations, the system may generate the reconstructed volume from the inputs utilizing an algorithm (e.g., PM2vector algorithm) to convert the projection matrices in camera format to vector variables (e.g., in the ASTRA toolbox). Another algorithm may be the same as or similar to the ASTRA FDK Recon algorithm that may call an FDK (Feldkamp, Davis, and Kress) reconstruction module, where normalized projection images may be cosine weighted and ramp filtered, then back-projected to the volume according to the cone-beam geometry. In some cases, an algorithm may be employed to convert the reconstructed volume (as output from the ASTRA FDK Recon algorithm, for example) in an appropriate format. For example, a NifTI processing algorithm may save the reconstructed volume as a NifTI image with an affine matrix.

Referring back to FIG. 6, in some cases, the pose may be estimated using an external imaging device. For example, an optical marker 633 (visible in camera view) may be placed on the C-arm or imager while a camera may be located at the robotic cart 620. A pose of the imager may be estimated based on images acquired by the camera from multiple viewpoints using computer vision methods. Alternatively or additionally, an optical marker 635 may be placed on a body part of the patient and/or at the patient bed 631 while the camera may be placed on the C-arm or the imager 603 such that the pose of the imager may be estimated based on images acquired by the camera when it moves with the C-arm. The camera may be a grayscale camera, color camera, depth camera, time-of-flight camera, a combination of depth and grayscale camera or a combination of depth and color camera. The pose estimation algorithm can be the same as those described above.

In some cases, poses estimated using different methods/fiducial landmarks may be combined to reduce the ambiguity and/or to improve the accuracy of the pose estimation. For example, a first pose may be reconstructed using a first 3D marker placed on the patient, a second pose may be reconstructed using the anatomical (e.g., rib) image as a second marker, and a final pose may be estimated based on the first pose and the second pose. In some cases, the final pose may be a fusion of the two poses estimated using the different fiducial landmarks (e.g., average). For example, the final pose may be a weighted average of two or more poses that takes into account the varying confidence of the estimation or the image quality. For instance, one or more 2D fluoroscopic images may be acquired which contain the image of both the rib image and the 3D artificial marker. A first pose may be estimated utilizing the rib image and a second pose may be estimated based on the 3D artificial marker, and a final pose of the camera may be calculated based on a weighted average of the first pose and the second pose. Alternatively, the first pose may serve as an initial solution to an optimization problem to use image alignment to calculate a refined second pose. For instance, the optimization algorithm (e.g., be a least squares algorithm, such as the global Levenberg-Marquardt optimization) as described above may be used to refine camera calibration parameters by minimizing the reprojection error where the first pose estimated based on the rib image may be used as the initial solution and the second pose estimated based on the artificial 3D marker may be used to refine the calibration parameters.

In some cases, pre-image processing or post-imaging processing may be performed to further enhance the 3D image quality. For example, 2D images may be aligned, normalized prior to reconstructing the 3D image as described above.

In some cases, a final pose estimation may be generated based on estimations made using different techniques and/or a fusion of different types of input data. For example, a final pose may be estimated based on a first pose measured by an inertial measurement units (IMUs), and a second pose estimated using the 3D marker (e.g., artificial 3D marker, bone structure/natural landmarks or a combination of both) as described above. In some cases, the system may employ a deep learning framework that can automatically adapt to the available inputs (e.g., IMU data, location sensor, computer vision based pose estimation, etc.) to generate a final pose estimation.

In some cases, the pose estimation method may include combining the multiple sensing modalities using a unique fusion framework. For instance, the framework may combine IMU sensor data, data from camera i.e., direct imaging device, image data from tomosynthesis, and the like to predict a pose. In some cases, the multiple input sources may be dynamically fused based on a real-time confidence score or uncertainty associated with each input source.

The intelligent fusion framework may include one or more predictive models can be trained using any suitable deep learning networks as described above. The deep learning model may be trained using supervised learning or semi-supervised learning. For example, in order to train the deep learning network, pairs of datasets with input image data (i.e., images captured by the camera) and desired output data (e.g., pose of tomosynthesis imager) may be generated by a training module of the system as training dataset.

Alternatively or in addition to, hand-crafted rules may be utilized by the fusion framework. For example, a confidence score may be generated for each of the different inputs and the multiple data may be combined based on the real-time condition.

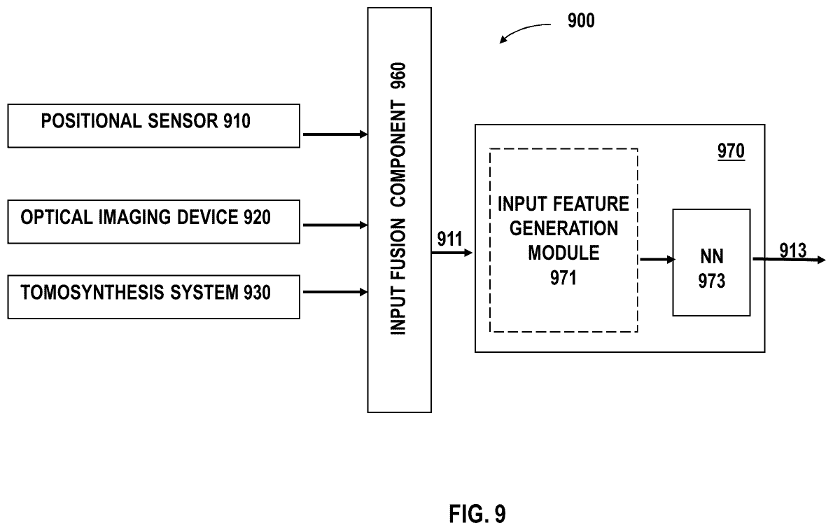

FIG. 9 schematically illustrates an intelligent fusion framework 900 for dynamically fusing and processing real-time sensory data to generate a pose estimation for the imaging device. In some embodiments, the intelligent fusion framework 900 may comprise a motion/location sensor (e.g., IMU) 910, an optical imaging device (e.g., camera) 920 for imaging 3D markers, a tomosynthesis system 930 for imaging radiopaque markers (e.g., 3D artificial marker or natural landmarks), an input fusion component 960 and an intelligent pose inference engine 670.

In some embodiments, the output 913 of the pose inference engine 670 may include a final estimated pose of the fluoroscopic imager. In some embodiments, the pose inference engine 670) may include an input feature generation module 971 and a trained predictive model 973. A predictive model may be a trained model or trained using machine learning algorithm. The machine learning algorithm can be any type of machine learning network such as: a support vector machine (SVM), a naïve Bayes classification, a linear regression model, a quantile regression model, a logistic regression model, a random forest, a neural network, convolutional neural network CNN, recurrent neural network RNN, a gradient-boosted classifier or repressor, or another supervised or unsupervised machine learning algorithm (e.g., generative adversarial network (GAN), Cycle-GAN, etc.).

The input feature generation module 971 may generate input feature data to be processed by the trained predictive model 973. In some embodiments, the input feature generation module 971 may receive data from the positional sensor 910, optical imaging device (e.g., camera) 920, a tomosynthesis system 930, extract features and generate the input feature data. In some embodiments, the data received from the positional sensor 910, optical imaging device (e.g., camera) 920, tomosynthesis system 930, may include raw sensor data (e.g., visible image data, fluoroscopic image data, IMU data, etc.). In some cases, the input feature generation module 971 may pre-process the raw input data (e.g., data alignment) generated by the multiple different sensory systems (e.g., sensors may capture data at different frequency) or from different sources (e.g., third-party application data) such as by aligning the data with respect to time and/or identified features (e.g., markers). In some cases, the multiple sources of data may be captured concurrently.

The data received from the variety of data sources 910, 920, 930 may include processed data. For example, data from the tomosynthesis system may include reconstructed data or pose estimated based on radiopaque markers.

In some cases, the data 911 received from the multimodal data sources may be adaptive to real-time conditions. The input fusion component 960 may be operably coupled to the data sources to receive the respective output data. In some cases, the output data produced by the data sources 910, 920, 930 may be dynamically adjusted based on real-time conditions. For instance, the multiple data sources may be dynamically fused based on a real-time confidence score or uncertainty associated with each data source or availability. The input fusion component 960 may assess the confidence score for each data source and determine the input data to be used for estimating the pose. For example, when a camera view is blocked, or when the quality of the sensor data is not good enough to identify a 3D marker, the corresponding input source may be assigned a low confidence score. In some cases, the input fusion component 960 may weight the data from the multiple sources based on the confidence score. The multiple input data may be combined based on the real-time condition.

In alternative cases, the framework 900 may be capable of adapting to different available input data sources. Instead of training multiple models corresponding to different input/output combinations, the framework 900 may allow for predicting a final pose regardless the available input data sources. In some cases, this may be achieved by synthesizing a missing input data base based on currently available input data. For instance, when only IMU data and tomosynthesis data are available, the input feature generation module 971 may first synthesize the missing visible image data based on the IMU data and tomosynthesis data then generate the input feature to be processed by the neural network 973.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for navigating a robotic endoscopic apparatus comprising:
(a) acquiring one or more fluoroscopic images using a fluoroscopic imager, wherein the one or more fluoroscopic images contain an image of a fiducial marker, a body part and a portion of the robotic endoscopic apparatus placed inside of the body part, wherein the fiducial marker is a three-dimensional (3D) marker;
(b) estimating a pose of the fluoroscopic imager based at least in part on an image of the fiducial marker in the one or more fluoroscopic images; and
(c) reconstructing a 3D fluoroscopic image based at least in part on the pose estimated in (b).

2. The method of claim 1, wherein the fiducial marker is deployed to a surgical field and wherein at least one of the one or more fluoroscopic images is acquired at an angle that is substantially aligned to a patient bed in the surgical field.

3. The method of claim 1, wherein the fiducial marker is an anatomical structure in the body part.

4. The method of claim 3, wherein the fiducial marker is a bone structure.

5. The method of claim 1, wherein the robotic endoscopic apparatus is disposable.

6. The method of claim 1, further comprising confirming the portion of the robotic endoscopic apparatus is inside a target tissue in the body part based on the 3D fluoroscopic image.

7. The method of claim 1, further comprising updating a location of a target tissue based on the 3D fluoroscopic image.

8. The method of claim 1, wherein the fiducial marker comprises a first 3D marker and a second 3D marker.

9. The method of claim 8, wherein the pose of the fluoroscopic imager is estimated based on a first pose estimated using the first 3D marker and a second pose estimated using the second 3D marker.

10. The method of claim 9, wherein the pose of the fluoroscopic imager is a weighted average of the first pose and the second pose.

11. The method of claim 9, wherein the pose of the fluoroscopic imager is estimated using an optimization algorithm and wherein the first pose or the second pose is used as an initial solution of the optimization algorithm.

12. The method of claim 9, wherein the first fiducial marker is an anatomical structure and the second fiducial marker is an artificial 3D marker.

13. The method of claim 1, wherein the fiducial marker includes a first component located at a patient bed and a second component located on a patient.

14. The method of claim 1, further comprising receiving a measured pose of the fluoroscopic imager based on location sensor data and fusing the measured pose with the pose estimated in (b).

15. A non-transitory computer-readable storage medium including instructions that, when executed by one or more processors, cause the one or more processors to perform operations comprising:
(a) acquiring one or more fluoroscopic images using a fluoroscopic imager, wherein the one or more fluoroscopic images contain an image of a fiducial marker, a body part and a portion of the robotic endoscopic apparatus placed inside of the body part, wherein the fiducial marker is a three-dimensional (3D) marker;
(b) estimating a pose of the fluoroscopic imager based at least in part on an image of the fiducial marker in the one or more fluoroscopic images; and
(c) reconstructing a 3D fluoroscopic image based at least in part on the pose estimated in (b)

16. The non-transitory computer-readable storage medium of claim 15, wherein the fiducial marker is deployed to a surgical field and wherein at least one of the one or more fluoroscopic images is acquired at an angle that is substantially aligned to a patient bed in the surgical field.

17. The non-transitory computer-readable storage medium of claim 15, wherein the fiducial marker is a bone structure.

18. The non-transitory computer-readable storage medium of claim 15, wherein the fiducial marker comprises a first 3D marker and a second 3D marker.

19. The non-transitory computer-readable storage medium of claim 18, wherein the pose of the fluoroscopic imager is estimated based on a first pose estimated using the first 3D marker and a second pose estimated using the second 3D marker.

20. The non-transitory computer-readable storage medium of claim 19, wherein the pose of the fluoroscopic imager is a weighted average of the first pose and the second pose.
